(19) United States
(12) Patent Application Publication
(10) Pub. No.: US 2024/0233870 A9
(43) Pub. Date: Jul. 11, 2024

(54) ASSOCIATIVE PROCESSING MEMORY SEQUENCE ALIGNMENT

(71) Applicant: MICRON TECHNOLOGY, INC., BOISE, ID (US)

(72) Inventors: Justin Eno, Boise, ID (US); Sean S. Eilert, Boise, ID (US); Ameen D. Akel, Boise, ID (US); Kenneth M. Curewitz, Boise, ID (US)

(73) Assignee: MICRON TECHNOLOGY, INC., Boise, ID (US)

(21) Appl. No.: 18/049,506

(22) Filed: Oct. 25, 2022

(51) Int. Cl. G16B 30/10; G16B 50/00

(52) U.S. Cl. CPC G16B 30/10; G16B 50/00

(57) ABSTRACT

Associative processing memory (APM) may be used to align reads to a reference sequence. The APM may store shifted permutations and/or other permutations of the reference sequence. A read may be compared to some or all of the permutations of the reference sequence and the APM may provide an output for each comparison. In some examples, the APM may compare the read to many permutations of the reference sequence to the read in parallel. Inferences may be made based on the comparisons between the read and the portions and/or permutations of a reference sequence. Based on the inferences, a candidate alignment location in the reference sequence for a read to be determined.

### BACKGROUND

Genetic information of an organism is stored in a genome which includes linear strings (e.g., sequences) of bases, referred to as nucleotides, which encode all of the instructions necessary for the organism. Common examples include deoxyribonucleic acid (DNA), which includes nucleotides adenine (A), guanine (G), cytosine (C), and thymine (T); and ribonucleic acid (RNA), which includes nucleotides A, C, G, but instead of T includes uracil (U). Determining the order of the nucleotides in the genome (e.g., the sequence), or portions thereof, is referred to as sequencing.

Determining a sequence of genetic information (e.g., a random DNA fragment, a gene, chromosome, entire genome) involves breaking the string of nucleotides into shorter strings and amplifying (e.g., replicating) the shorter strings. The sequences of the shorter strings are then determined, such as by tagging different nucleotides with different fluorescent markers and analyzing the fluorescent signals. However, other techniques for sequencing exist. Each sequence determined for a shorter string is referred to as a “read.” These reads are analyzed and recombined (e.g., aligned) to provide the sequence of the longer string (e.g., the sequence of genetic information). In some cases, the reads may be aligned de novo to determine an unknown genetic sequence. In other cases, the reads may be aligned to a reference sequence.

When a reference sequence is used, the reads from a sample sequence are compared to the reference sequence to determine where the reads align to the reference sequence (e.g., alignment location). That is, at what location along the reference sequence the nucleotides of the read match the nucleotides of the reference sequence (if any). The reads may be aligned into the sample sequence based on where in the reference sequence the matches occurred. Or, if few or none of the reads had matches, it could be determined that the reads were from a sample that did not match the reference sequence. For example, a sample may be acquired from a patient, and reads from the sample may be compared to one or more reference sequences of one or more known pathogens (e.g., virus, bacteria). Based on the comparison of the reads to the reference sequences, it may be determined whether the patient is infected with one of the known pathogens. Thus, in some applications, aligning reads to a reference sequence may be used for diagnostic purposes.

Sequencing technologies, particularly next generation sequencing (NGS) systems, generate millions to billions of reads ranging anywhere from less than fifty nucleotides to more than a thousand nucleotides. Aligning these millions to billions of reads requires significant computation time. Accordingly, improved alignment techniques are desired.

### BRIEF DESCRIPTION OF THE DRAWINGS

FIG. 1 illustrates an example of a system for in-memory associative processing in accordance with examples as disclosed herein.

FIG. 2 illustrates at least a portion of an associative processing memory device in accordance with examples disclosed herein.

FIG. 3 illustrates certain functions of an associative processing memory device in accordance with examples disclosed herein.

FIG. 4 illustrates an example of storing a reference sequence in an associative processing memory in accordance with examples disclosed herein.

FIG. 5 illustrates an example of comparing reads to reference sequences stored in the associative processing memory shown in FIG. 4 in accordance with examples disclosed herein.

FIG. 6 is a table of results of comparisons of the reads to the reference sequence shown in FIG. 5 in accordance with examples disclosed herein.

FIG. 7 is a flowchart illustrating inferences based on results for a read provided by an associated processing memory device in accordance with examples disclosed herein.

### DETAILED DESCRIPTION

As disclosed herein, associative processing memory (APM) may be used to align reads to a reference sequence. In some embodiments, the APM may store shifted permutations and/or other permutations of the reference sequence. A read may be compared to some or all of the permutations of the reference sequence in the APM. The APM may provide a result for each comparison which indicates how well the read matched that version of the reference sequence (e.g., how many nucleotides matched between the read and the reference sequence). In some examples, the APM may compare the read to many permutations of the reference sequence to the read in parallel, which may reduce computation time in some applications.

In some embodiments, inferences may be made based on the comparisons between the read and the portions and/or permutations of a reference sequence (e.g., the results provided by the APM). For example, whether the read has an insertion or deletion error may be inferred in some cases. In another example, whether the read has a transcription error may be inferred. The inference of an error may permit a candidate alignment location (or lack thereof) in the reference sequence for a read to be determined. This may improve tolerance for read errors and/or mutations in some applications.

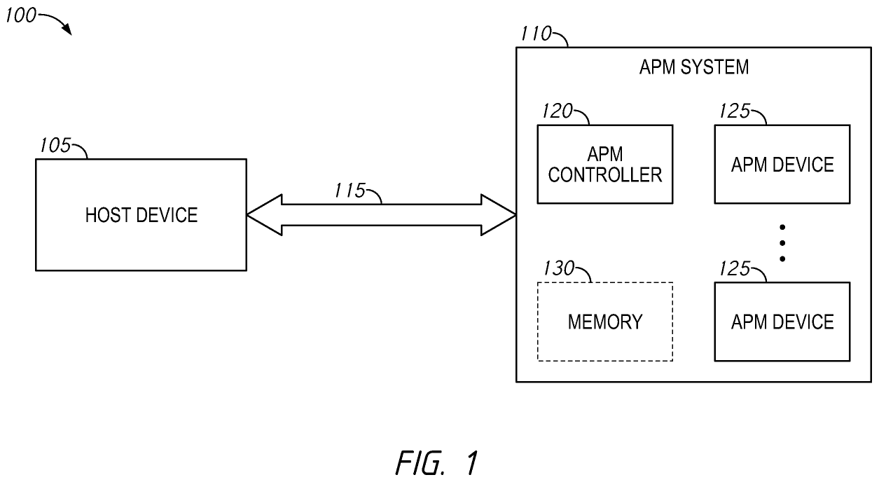

FIG. 1 illustrates an example of a system 100 for in-memory associative processing in accordance with examples as disclosed herein. The system 100 may include a host device 105 and an associative processing memory (APM) system 110. The host device 105 may interact with (e.g., communicate with, control) the APM system 110 as well as other components of the device that includes the system 100. In some examples, the host device 105 and the APM system 110 may interact over the interface 115, which may be an example of a Compute Express Link (CXL) interface or other type of interface.

In some examples, the system 100, or a portion thereof, may be included in, or coupled with, a computing device, an electronic device, a mobile computing device, or a wireless device. The device may be a computer, a laptop computer, a tablet computer, a smartphone, a cellular phone, a wearable device, an internet-connected device, or the like. In some examples, the system 100 may be included in a system for genetic sequencing, such as a NGS system. In some examples, the host device 105 may be included in a different device from the APM system 110. For example, host device 105 may be included in a genetic sequencing system and the APM system may be included in a separate computing device in communication with the genetic sequencing system. In some examples, the system 100 may be included in one or more computing devices in communication with a genetic sequencing system (e.g., via host device 105). The host device 105 may be or include a system-on-a chip (SoC), a general-purpose processor, a digital signal processor (DSP), an application-specific integrated circuit (ASIC), a field-programmable gate array (FPGA) or other programmable logic device, discrete gate or transistor logic, discrete hardware components, or it may be a combination of these types of components. In some examples, the host device 105 may be referred to as a host, a host system, or other suitable terminology.

The APM system 110 may operate as an accelerator (e.g., a high-speed processor) for the host device 105 so that the host device 105 can offload various processing tasks to the APM system 110. For example, the device 105 may send a program (e.g., computer-readable/processor or controller executable instructions) to the APM system 110 for execution by the APM system 110. As part of the program, or as directed by the program, the APM system 110 may perform various computational operations such as comparing reads taken from a sample of genetic material to a reference sequence. In some examples, the program may be stored in memory 130. In some examples, memory 130 may include a non-transitory computer-readable medium.

The APM controller 120 may be configured to interface with the host device 105 on behalf of the APM devices 125. Upon receipt of a program from the host device 105 (or retrieval of the program from memory 130), the APM controller 120 may parse the program and direct or otherwise prompt the APM devices 125 to perform various computational operations associated with or indicated by the program. In some examples, the APM controller 120 may retrieve (e.g., from the memory 130) the reference sequence and/or reads for the computational operations and may communicate the reference sequence and/or reads to the APM devices 125 for associative processing. In some examples, the APM controller 120 may indicate the reads and/or reference sequence for the computational operations to the APM devices 125 so that the APM devices 125 can retrieve the reads and/or reference sequence from the memory 130. The memory 130 may be configured to store reads and sequences that are accessible by the APM controller 120, the APM device 125, the host device 105, or a combination thereof. In some examples, the host device 105 may provide the reads and/or reference sequence to the APM system 110. In some embodiments, the host device 105 may provide the reads and/or reference sequence to the APM controller 120 and/or the memory 130. Although shown included in the APM system 110, the memory 130 may be external to, but nonetheless coupled with, the APM system 110. Although shown as a single component, the functionality of memory 130 may be provided by multiple memories 130.

The reads and/or reference sequence for analysis by the APM devices 125 may be indicated by (or accompanied by) the program received from the host device 105 or by other control signaling (e.g., other separate control signaling) associated with the program. For example, a program may indicate how the reads and/or reference sequences should be stored and/or provided to the APM devices 125. In some examples, the program may indicate variations (e.g., sequence shifting, padding, and/or other permutations) to the reads and/or reference sequences to be provided and/or stored in the APM devices 125.

The APM devices 125 may provide ternary content-addressable memory (TCAM) functionality. The APM may include memory cells, such as content-addressable memory (CAM) cells. Examples of CAM and CAM cells are described in U.S. Pat. Nos. 9,934,856, 10,068,652, 10,210,911, and 11,264,096, which are incorporated herein by reference for any purpose. However, any suitable CAM and CAM cell structure may be used. In some embodiments, CAM cells may provide TCAM functionality. In some embodiments, the CAM cells may interact with other components of the APM devices 125 to provide the TCAM functionality. The memory cells may be organized as an array of rows and columns.

At least some APM devices 125, if not each APM device 125, may use associative processing to perform computational operations on the data stored in that APM device 125. Unlike serial processing (where vectors are moved back and forth between a processor and a memory), associative processing may involve searching and writing data in-memory (also referred to as “in-situ”), which may allow for parallelism that increases processing bandwidth. Performance of computational operations in-situ may also allow the system 100, among other advantages, avoid the bottleneck at the interface between the host device 105 and the APM system 110, which may reduce latency and power consumption compared to other processing techniques, such as serial processing. Associative processing may also be referred to as associative computing or other suitable terminology.

The associative processing techniques described herein may be implemented by logic at the APM system 110, by logic at the APM devices 125, or by logic that is distributed between the APM system 110 and the APM devices 125. The logic may include one or more controllers, access circuitry, communication circuitry, or a combination thereof, among other components and circuits. The logic may be configured to perform aspects of the techniques described herein, cause components of the APM system 110 and/or the APM devices 125 to perform aspects of the techniques described herein, or both.

Use of multiple APM devices 125, as opposed to a single APM device 125, may further increase the bandwidth of the APM system 110 relative to other systems. Each APM device 125 may include a local controller and/or logic that controls the operations of that APM device 125.

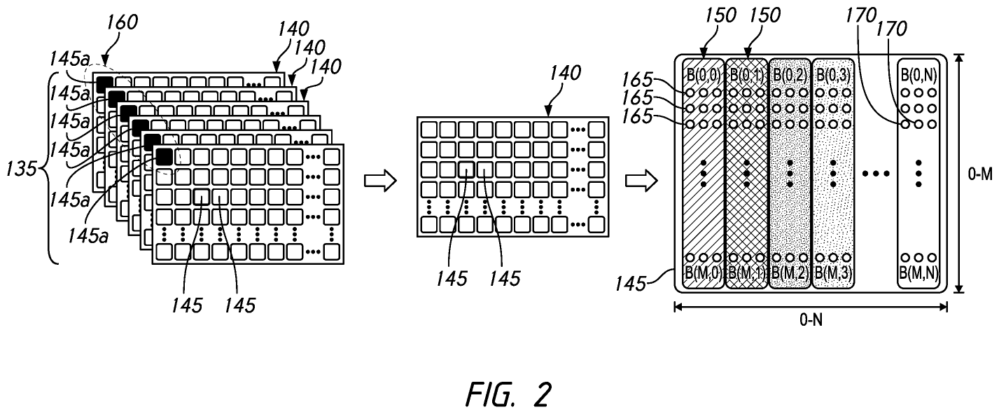

FIG. 2 illustrates at least a portion of an associative processing memory device in accordance with examples disclosed herein. The APM device shown in FIG. 2 may be used to implement one or more of the APM devices 125 in some examples. The APM device may include one or more dies 135, which may also be referred to as memory dies, semiconductor dies, or other suitable terminology. A die 135 may include one or more tiles 140, which in turn may each include one or more planes 145. In some examples, the tiles 140 may be configured such that a single plane 145 per tile is operable or activatable at a time (e.g., one plane per tile may perform associative computing at a time). However, any quantity of tiles 140 may be active at a time (e.g., any quantity of tiles may be performing associative computing at a time). Thus, the tiles 140 may be operated in parallel, which may increase the quantity of computational operations that can be performed during a time interval, which in turn may increase the bandwidth of an APM device relative to other different techniques. When a plane 145 is activated in each tile 140, the set of activated planes 145 may be referred to as a hyperplane. An example of a hyperplane 160 is shown as the set of activated planes 145a. While the example of the hyperplane 160 includes active planes 145a that are located in a same location for each tile 140, hyperplane 160 is not limited to this arrangement (e.g., one or more of the active planes 145a may be in different locations in the tiles 140 from other active planes 145a).

Each plane 145 may include a memory array that includes memory cells 170, which may be CAM cells in some examples. The memory cells 170 in a memory array may be arranged in columns and rows and may be non-volatile memory cells or volatile memory cells. A memory array that includes CAM cells may be configured to search the CAM cells by content as opposed to by address. For example, a memory array that includes CAM cells storing a truth table for a computational operation may compare the logic values of operand bits with the content of the CAM cells to determine which results correspond to those logic values.

As noted, an APM device 125 may be configured to store data associated with genetic information (e.g., nucleotides in a string) in the memory cells of that APM device 125. To aid in associative processing, the data may be stored in a columnar manner across a portion of a plane 145 or across multiple planes 145. For example, a read, reference sequence, or portion thereof may be stored in one or more columns 150 of one or more planes 145. For example, each row 165 of each column 150 B(0-M,0-N) shown in FIG. 2 may include memory cells 170 that store data correspond to a different nucleotide in one or more reads or sequences stored in the plane 145. In some examples, the width (e.g., the number of memory cells 170) of each column 150 may correspond to a number of bits used to encode the nucleotides of the sequence. For example, DNA and RNA both consist of four nucleotides. Thus, in some examples, two bits may be used to represent the nucleotides. As will be described in more detail, additional information may be included in the sequences, such as an indication of a “don't care” position in the sequence. In these examples, additional bits may be used (e.g., three bits, four bits) to represent the nucleotides. For example, when the genetic sequence is a DNA sequence, adenine=000, guanine=001, cytosine=010, thymine=011, and don't care=100. This example is provided for explanatory purposes, and the number and value of bits assigned to different nucleotides and/or “don't care” may be different in other examples.

In some examples, in addition to or instead of indicating “don't care” positions in the sequence in the memory cells, the “don't care” indication may be provided by the APM device by providing a mask of “don't care” positions that ignore the masked memory cells 170. By ignore, it means that data provided by the masked memory cells may not be used to compute a result. In these examples, the masked memory cells 170 may be programmed with dummy values or the existing data stored in the masked memory cells 170 may remain unchanged.

Each bit may be stored in a separate memory cell 170 of the column 150 in some examples. For example, the data representing a nucleotide B(0,0) may be stored as bits in two or more memory cells 170. The number of columns 150 N may be based, at least in part, on a size of the plane 145 and/or a width of each column 150 in some examples. The number of rows 165 M in each column 150 N may be based, at least in part, on a size of the plane 145 in some examples. In some examples, a plane 145 may be 256×256 bits, and each tile 140 may include 1,024 planes 145, and a die 135 may include 4,096 tiles 140. However, the disclosure is not limited to these particular sizes.

Reads and/or reference sequences may be stored in a variety of arrangements in the APM device. For example, for short sequences (either reads or reference sequences), a sequence may be stored in a single column 150. For longer sequences, the sequences may be stored across multiple columns 150 of a single plane 145 or multiple planes 145. The planes 145 may be on the same tile 140 and/or on different tiles 140.

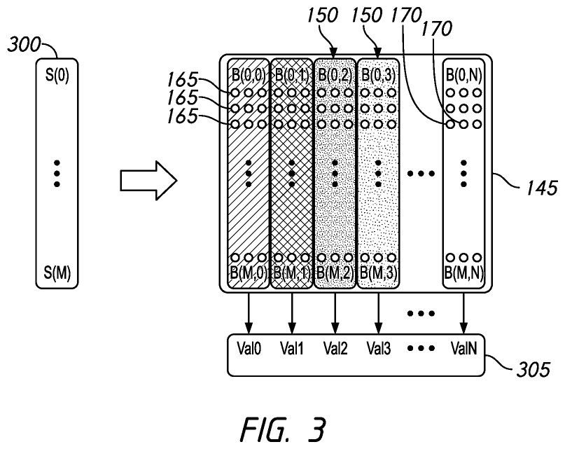

FIG. 3 illustrates certain functions of an associative processing memory device in accordance with examples disclosed herein. One of the functions for which APM including a CAM may be used is for is determining whether data is stored in the CAM. As shown in FIG. 3, input data 300 may be compared to the data stored in the plane 145, and a result 305 indicating whether data matching input data 300 is located in the plane 145 may be provided.

In the example shown in FIG. 3, data 300 is a column of data S(0,M). The column of data 300 may have the same width and number of rows as columns 150. In some examples, the data 300 may be compared to all of the columns 150 in plane 145 in parallel (e.g., substantially at the same time). In the example shown in FIG. 3, the result 305 may include multiple values Val0-ValN, where the number of values is equal to the number of columns 150 N in the plane 145. In some examples, the values Val0-ValN may be binary (e.g., 0=not all of the data 300 match the data in the column, 1=all of the bits in data 300 match the data in the column). In some examples, the values Val0-ValN may have values ranging from 0-M, where M is the number of rows 165 in the columns 150. The value may indicate the number of rows in data 300 where the bits match the bits in the corresponding row 165 of the column 150 (e.g., a score). For example, Val0=0 if there are no matches between B(0,0)-B(M,0) and S(0-M) and Val0=M if the data in all of the rows match between the columns. In some examples, the values Val0-ValN may have a value representing a Hamming distance between B(0,0)-B(M,0) and S(0-M).

In some examples, the CAM may be a ternary CAM. In some examples, this may allow the CAM to accommodate “do not care” entries in the data. As noted previously, bits may be used to store the different nucleotides of a genetic sequence (e.g., DNA, RNA). In some applications, at certain locations it may not matter whether or not two nucleotides match. For example, reads and/or reference sequences may be of varying lengths. In order to accommodate the different lengths in the APM device, the reads and/or reference sequences may be “padded” with “do not care” entries in one or more rows to make all of the reads and/or reference sequences the same length. In other examples, “do not care” entries may allow shifted reads and/or sequences to be stored in the APM device. In some examples, when a row of data 300 and/or a row of a column 150 indicates a “do not care” data, the result 305 may indicate a match for that row of column 150 regardless of the bits in the row of data 300 and/or row of column 150.

In some examples, the reads and/or reference sequences may be padded with dummy values that achieve the “do not care” functionality. In some examples, when the APM device provides the reads, the APM device may mask the memory cells corresponding to “do not care values” in the read and/or reference sequences. The data from these memory cells may be ignored. For example, when S(0) is provided for comparison to sequences in the columns 150, results from memory cells 170 compared to the dummy values in S(0) may be ignored (regardless of whether the memory cells 170 are masked) and/or results from memory cells 170 that are masked may be ignored (regardless of whether the corresponding value of S(0) includes a dummy value). Providing or storing a “don't care” value, a dummy value, or masking a memory cell may be collectively described in terms of a “don't care” value, but it should be understood that the effect may be achieved by one or more of these techniques.

In some examples, a computing device (e.g., a sequencing system or other computing system) may perform a quality assessment of a read and/or a reference sequence. The quality assessment may provide a quality score for each nucleotide of the sequence. The quality score may represent a determination of an expected accuracy of a nucleotide of the sequence. In some examples, if the quality score is low, meaning the expected accuracy is low (e.g., equal to or below a threshold value), rather than providing the read nucleotide, a “don't care” value may be provided at the location of the nucleotide in the sequence.

According to embodiments of the present disclosure, data 300 may include a read, and one or more planes 145 may include one or more reference sequences (and/or permutations of one or more reference sequences). The read may be compared to all of the columns of a plane 145 in parallel. The read may be compared to all of the planes 145a of a hyperplane 160 in parallel. In examples, the read may be compared to reference sequences (and/or permutations of one or more reference sequences) stored in multiple APM devices (e.g., APM devices 125) in parallel. Alternatively, data 300 may include a reference sequence (or a portion or a permutation thereof) and one or more planes 145 may include one or more reads. In some applications, this may increase parallel searching capabilities for matches between reads and reference sequences. In some applications, this may reduce computation time.

Ideally, each read from a longer sequence (e.g., a sequence extracted from a sample from an organism) would be compared to all portions of the reference sequence, and a match would indicate where in the reference sequence the read corresponded to (e.g., aligned). Once all the reads had been processed by the APM, the reads could be aligned into the longer sequence based on where in the reference sequence the match occurred. Or, if few or none of the reads had matches, it could be determined that the reads were from a sequence that did not match the reference sequence. For example, reads from a viral sequence compared to a SARS-CoV-2 reference sequence may not match if the viral sequence was from an influenza virus.

However, aligning reads to a reference sequence is usually not that straight forward. Typically, a genomic sample from which reads are obtained does not have a sequence that exactly matches a reference sequence, even if the genomic sample is a same organism. For example, mutations may cause changes in the sequence between the sample and the original organism from which the reference sequence was obtained (e.g., alpha, beta, delta, and omicron variants and sub-variants thereof for SARS-CoV-2). Additionally, the process of obtaining reads from the sample is not perfect. Some reads may include one or more of a mismatched nucleotide (e.g., a transcription error), a deletion of a nucleotide and/or an insertion of a nucleotide. The inventors of the present disclosure have recognized that by comparing the reads to permutations of the reference sequence, whether a read error has occurred may be inferred. In some applications, this may allow alignment of at least some reads to the reference sequence, even if those reads contain errors or mutations. Furthermore, the techniques disclosed herein may take advantage of the structure of the APM, permitting both improved processing speed by utilizing the mass parallel processing capabilities of the APM and reducing downstream processing by finding candidate alignment positions of reads despite the presence of errors.

In some embodiments, one or more APM devices (e.g., APM devices 125) may store shifted permutations (e.g., versions) of a reference sequence. A read may be compared to some or all of the permutations of the reference sequence in the APM device. The APM device, or a portion of the APM device, may provide an output for each comparison which indicates how well the read sequence matched that version of the reference sequence (e.g., how many nucleotides matched between the read and the reference sequence). For example, each plane 145 may provide a result 305.

In some embodiments, using the output (e.g., the one or more results 305), inferences may be made based on which permutation or permutations of the reference sequence the read matched with the best (e.g., column 150 with the most matches) and/or worst (e.g., column 150 with the least matches). For example, whether the read has an insertion or deletion error may be inferred in some cases. In another example, whether the read has a mutation (or a nucleotide transcription error) may be inferred. This may improve tolerance for read errors and/or mutations in some applications.

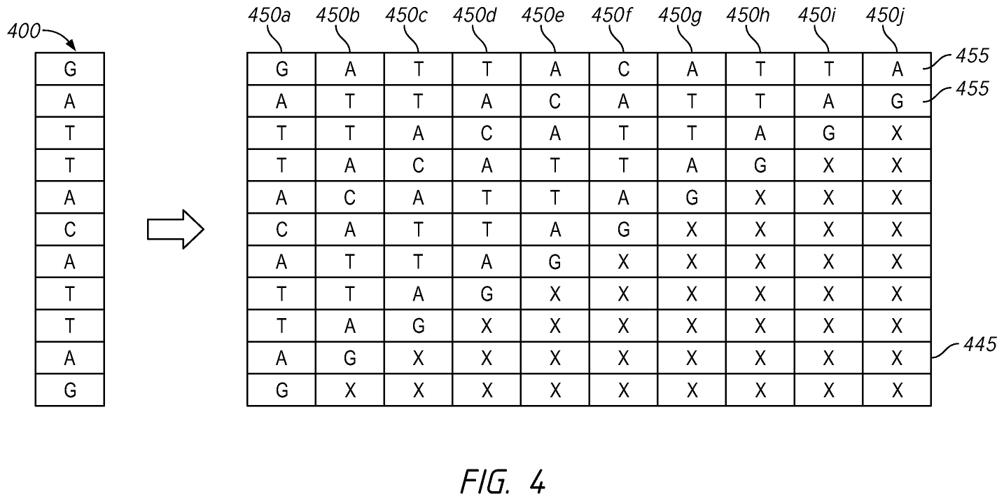

FIG. 4 illustrates an example of storing a reference sequence in an associative processing memory in accordance with examples disclosed herein. For ease of illustration, only one plane 445 of an APM device is shown in FIG. 4. Plane 445 may be included in APM device 125 in some examples. While plane 445 is illustrated as including ten columns 450a-j and eleven rows, it is understood that plane 445 may have more columns and rows in some examples. In some examples, FIG. 4 may show only a portion of plane 445. In some examples, plane 445 may be used to implement or be included in plane 145. Further, while each row of the columns 450a-j include one or more memory cells that store bits indicating a nucleotide of a genetic sequence, for ease of illustration, the nucleotides stored in each row of each column 450a-j is indicated by a block 455 with a letter. In the example shown in FIG. 4, adenine=A, guanine=G, cytosine=C, thymine=T, and X=don't care.

A reference sequence 400 may be provided to the APM device (e.g., via host device 105, APM controller 120, and/or memory 130) for storage. The APM device may store the original reference sequence 400 in column 450a, and in each subsequent column 450b-j shift the reference sequence by one nucleotide and appending a “don't care” to the end of the sequence for each shift (or applying a mask by the APM device). As shown, column 450b stores the reference sequence 400 shifted by one nucleotide and includes one “don't care” entry, column 450c stores the reference sequence 400 shifted by two nucleotides and includes two “don't care” entries, and column 450d includes the shifted by three nucleotides and includes three “don't care” entries, and so on. While not shown in FIG. 4, in some examples, the shifting of the reference sequence may continue across additional columns until the entirety of the sequence had been shifted. In other examples, the shifting may occur until the expected minimum read length is included in a column.

While the reference sequence 400 shown in FIG. 4 has the same number of rows as the columns 450a-j, in other examples, the reference sequence 400 may be longer and have more rows than the columns 450a-j. In these examples, the reference sequence 400 may be stored across multiple columns. When the reference sequence 400 may be longer and have more rows than columns 450a-j, in some examples, each column 450a-j may store a portion of the reference sequence 400, and each column 450a-j may include a shifted portion of the reference sequence 400. That is, the column length may act as a sliding window moving along the reference sequence 400 where each column 450a-j stores a portion of the reference sequence 400 at a different location of the sliding window. In some examples the sliding window may be shifted by one nucleotide at a time. In these examples, the masking and/or inclusion of “don't cares” within the columns 450a-j may not occur until the sliding window reaches the end of the reference sequence 400. For example FIG. 4 may be interpreted to show a portion of a reference sequence 400 that is longer than what is shown and a portion of plane 445 that is larger than what is shown, where the columns 450a-j store the end portion of the reference sequence 400 shown in FIG. 4. Thus, the “don't cares” may provide padding to resolve differences in dimensions between the reference sequence 400, the columns 450a-j, and/or read sequences.

In some examples, the columns storing the reference sequence 400 may be located in different planes and/or different tiles. In some examples, the reference sequence 400 may be padded with “don't cares,” even when not shifted, to make the reference sequence 400 a same length as a whole number of columns.

Furthermore, while referred to as a “reference sequence,” reference sequence 400 may be a portion of a longer reference sequence stored in one or more APM devices. For example, a longer reference sequence may be divided into smaller portions and each of the portions of the reference sequence (e.g., reference sequence 400 may be one of several portions) may be shifted as described with reference to FIG. 4. In some examples, the longer reference sequence may be divided into mutually exclusive portions. In other examples, the longer reference sequence may be divided into overlapping portions. That is, some of the nucleotides included in a portion may also be included in another portion. In some applications, dividing the longer reference sequence into overlapping portions may improve alignment of reads that include nucleotides from more than one portion.

The portions of the reference sequence and shifted permutations of the different portions of the reference sequence may be stored in different portions of an APM device and/or different APM devices of an APM system (e.g., APM devices 125 of APM system 110). For example, each portion of a reference sequence and the portion's shifted permutations may be stored in a separate plane (e.g., plane 145). In another example, each portion of the reference sequence and the portion's shifted permutations may be stored in a separate hyperplane (e.g., hyperplane 160). Other combinations of storing a reference sequence and its permutations may be used in other examples.

In some examples, how the reference sequence is divided into portions, stored, shifted, and/or padded is performed based on a program provided to an APM system (e.g., APM system 110) that includes the APM device. In some examples, the reference sequence 400 may be stored, shifted, and/or padded responsive to control signals by an APM controller, such as APM controller 120. The APM controller may provide the control signals responsive to the program in some examples.

Once the reference sequence (and/or portions of a longer reference sequence) and the shifted permutations of the reference sequence are stored in the APM device, reads acquired from a sample may be compared to the reference sequence by the APM device.

The reads may be provided to the APM device (e.g., APM device 125) by an APM controller (e.g., APM controller 120). In some examples, the APM controller may retrieve the reads from a memory (e.g., memory 130) and/or receive the reads from a host device (e.g., host device 105).

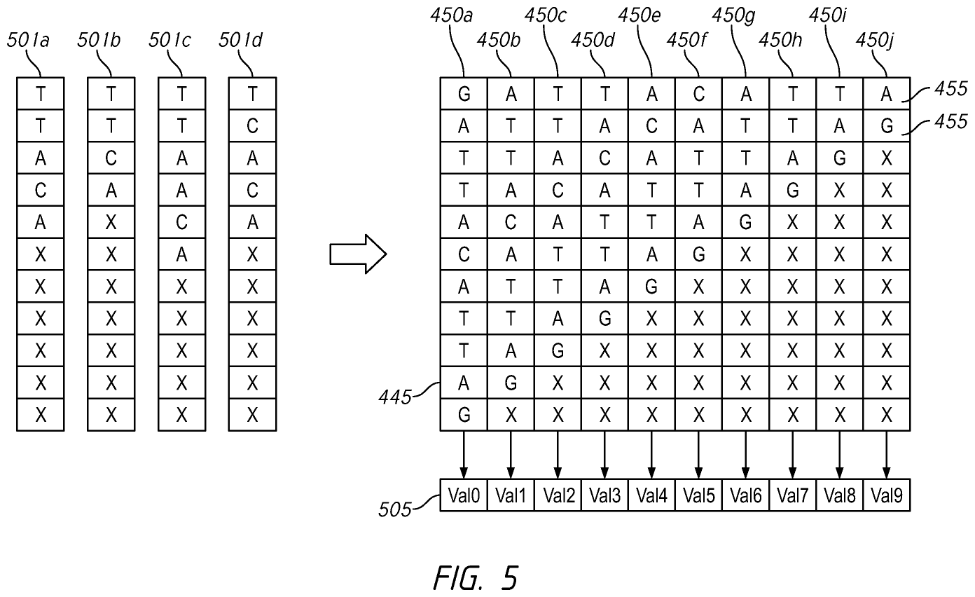

FIG. 5 illustrates an example of comparing reads to reference sequences stored in the associative processing memory shown in FIG. 4 in accordance with examples disclosed herein. Four example reads 501a, 501 b, 501c, 501d are shown. Read 501a is a “clean” read. That is, it accurately represents a portion of the sample sequence (not shown). Reads 501b-d are examples of erroneous reads. Read 501b is a read with a deletion error. That is, a nucleotide (A in the illustrated example) was omitted during the read process. Read 501c is a read with an insertion error, where a nucleotide (again, A in the illustrated example) is inserted into the sequence during the read process. Read 501d is a transcription error where the nucleotide in the second row has been replaced by a different nucleotide (T has been replaced with C). While described as a transcription error, the read 501d may be due to a mutation, that is, the sample sequence is a variant of reference sequence. However, for ease of illustration, read 501d will be referred to as an erroneous read. As shown in the example in FIG. 5, the reads 501a-d are shorter than the length of the reference sequence 400. In some examples, such as the one shown in FIG. 5, the reads 501a-d may be padded with “don't cares” to make the reads 501a-d the same length as the reference sequence 400 and/or columns 450a-j. In some examples, the “don't cares” may be dummy values. In some examples, the dummy values may indicate to the APM device to mask memory cells at corresponding locations when the reads 501a-d are compared to memory cells in the columns 450a-j.

In some examples, the reads 501a-d may be provided to the APM device in series for comparing to the reference sequence and permutations. In some examples, each of the reads 501a-d may be compared to multiple reference sequences (or portions of reference sequences when reference sequence 400 is a portion of a longer reference sequence) in parallel. For example, each read 501a-d may be compared to the reference sequence 400 and permutations in all of the columns of plane 445 in parallel. In some examples, each read 501a-d may be compared to reference sequences and/or permutations thereof located in different planes in parallel (e.g., hyperplane 160). In some examples, each of the reads 501a-d may be compared to multiple reference sequences stored in different APM devices in parallel (e.g. the multiple APM devices 125). For example, for COVID-19 testing, each APM device may include one or more SARS-CoV-2 variants or sub-variants. In some examples, two or more of reads 501a-d may be compared to reference sequences in parallel (e.g., different reads provided to different APM devices in parallel).

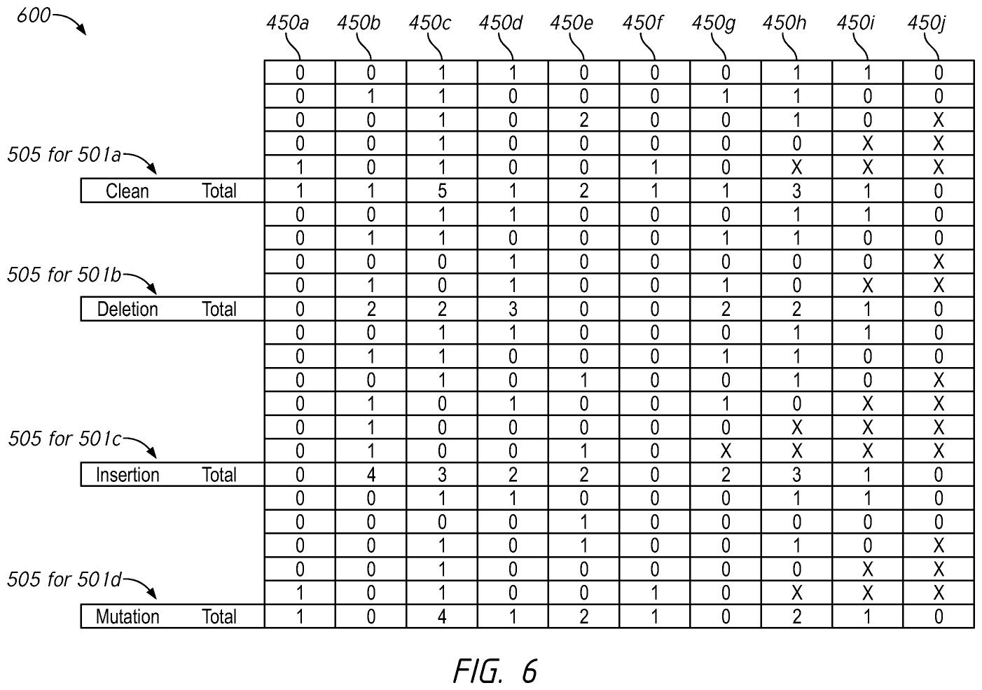

Based on the comparing, the APM device may provide one or more outputs. For example, plane 445 may provide a result 505 that includes values Val0-9. The number of values included in result 505 may be equal the number columns of plane 445. Similar to result 305, each of the values Val0-9 may indicate a number of matching nucleotides between the corresponding read 501a, 501b, 501c, or 501d and the columns 450a-j. In some examples, the values Val0-9 may indicate a Hamming distance between the read 501a, 501b, 501c, or 501d and the columns 450a-j. FIG. 6 is a table of results of comparisons of the reads to the reference sequence shown in FIG. 5 in accordance with examples disclosed herein. Based on the result 505, one or more inferences about the read may be made. In some examples, an inference may be made as to a location along the reference sequence that the read aligns to. In some examples, an inference may be made as to whether the read includes an error.

In the example shown in FIGS. 5-6, clean read 501a would have a “perfect” score for column 450c. That is, value Val2 may have a value that indicates that all of the nucleotides in the read 501a match all of the nucleotides in column 450c (which includes the reference sequence 400 shifted by two nucleotides). Because it is known which reference sequence (or portion thereof) is stored in the plane 445 and how much the reference sequence 400 is shifted in each column 450a-j, it can be determined where in the reference sequence 400 read 501a may be aligned to. The remaining values of the result 505 have lower scores, indicating the clean read 501a did not match all of the nucleotides stored in the remaining columns. Thus, a location of a clean read may be determined by the presence of a perfect score.

However, in the cases of erroneous reads, no perfect score may be present, and there may be clusters of columns (e.g., two or more) that include scores that indicate partial matches (e.g., only some, but more than zero, of the nucleotides between the reference sequence 400 and the read match). Deletion read 501b would not have a perfect score for any of the columns 450a-j, but would have a maximum score (e.g., the highest value from a column provided in the result 505) in column 450d (Val3 may have a value indicating three matching nucleotides) preceded by two lower scores in columns 450b and 450c (Val1 and Val2 may have values indicating two matching nucleotides).

Similarly, insertion read 501c would not have a perfect score for any of the columns 450a-j. However, in contrast to deletion read 501b, insertion read 501c has a maximum score in column 450b (Val1 may have a value indicating four matching nucleotides) followed by lower scores in columns 450c-e (Val2 may have a value indicating three matching nucleotides and Val3 and Val4 may have values indicating two matching nucleotides).

Transcription error read 501d has a near perfect score for column 450c (e.g., Val2 has a value indicating read 501d matches all but one nucleotide). While the score in column 450c is followed by a lower score for Val3 in column 450d, this score is significantly lower than the score for column 450c. This is in contrast for deletion read 501b and insertion read 501c which have clusters of columns with similar scores.

When no perfect score is present for any column, in some applications, it may be inferred when a read is a “match” except for an error in the read. In some embodiments, the relative scores and/or relative locations of the scores across the columns may be used to infer a location where an erroneous read may be aligned to the reference sequence 400.

When no perfect score is present for any column, a maximum score may be compared to a threshold value. In some examples, when a maximum score is equal to or greater than a threshold value (e.g., only one nucleotide in the column does not match), it may indicate a transcription error read that corresponds to the reference sequence 400 stored in the column of the maximum score. In the example shown in FIGS. 5 and 6, column 450c has the maximum score and the value of the score indicates that only one nucleotide in the read does not match the reference sequence 400. Accordingly, the “correct” location of the transcription error read is 450c, the same as for the clean read 501a. Optionally, further analysis may be performed based on the values of the columns adjacent to and/or near the column having the maximum score. For example, if the difference between the maximum score and the scores in the adjacent columns is greater than a threshold value (e.g., the adjacent columns have a difference in score greater than one or two), it may further indicate the read is a transcription error read.

In some examples, when the maximum score is less than the threshold value, when the maximum score located in a cluster is preceded by lower matching scores, it may indicate a deletion read that corresponds to the reference sequence 400 stored in the column preceding the column with the maximum score. In the example shown in FIGS. 5 and 6, column 450d has the maximum score, thus, the “correct” location of the deletion read 501b is column 450c, the same as for the clean read 501a. Optionally, further analysis may be performed based on the values of the columns adjacent to and/or near the column having the maximum score. For example, if the difference between the maximum score and the scores in the adjacent columns is less than a threshold value (e.g., the adjacent columns have a difference in score less than two), it may further indicate the read is a deletion read.

When no perfect score is present for any column, when a maximum score is followed by lower matching scores, it may indicate an insertion read that corresponds to the reference sequence 400 stored in the column following the column with the maximum score. In the example shown in FIGS. 5 and 6, column 450b has the maximum score, thus, the correct location of the insertion read 501c is column 450b, the same as for clean read 501a. Optionally, further analysis may be performed based on the values of the columns adjacent to and/or near the column having the maximum score. For example, if the difference between the maximum score and the scores in the adjacent columns is less than a threshold value (e.g., the adjacent columns have a difference in score less than two), it may further indicate the read is an insertion read.

When no perfect score is present for any column, in some examples, the maximum score may be compared to another threshold value. For example, if the maximum score is below the other threshold value, it may indicate that few or none of the nucleotides between the read and the reference sequence match. In this case, it may be determined that the read does not match the reference sequence 400, and no alignment location can be determined for the read. For example, if not enough nucleotides match between the read and the reference sequence, it may indicate the read corresponds to a sample that is a different organism from the reference sequence.

In some examples, additional clusters of columns including values indicating partial matches may be ignored if the values are less than the maximum score.

The example threshold values (e.g., a difference between the maximum and perfect score, a difference between scores of adjacent columns, a minimum number of nucleotides match) provided are based on one or two nucleotides, other threshold values may be used in other examples. In some embodiments, the threshold values may be varied as the length of the read increases. For example, a greater difference between the maximum score and a perfect score may be tolerated for determining a transcription error read, and/or a greater difference between the scores of adjacent or nearby columns may be tolerated for determining erroneous reads when the read is long. In some embodiments, the threshold values may be varied depending on a desired accuracy. For example, smaller differences between the maximum score and the perfect score and/or a greater minimum score may be used in applications where fewer errors in reads can be tolerated.

The inferences of the type of error (if any) in a read and/or location of the read in the reference sequence (e.g., location of alignment with the reference sequence) described with reference to FIGS. 5-6 may be determined based on the results (e.g., results 305, 505) provided by one or more planes (e.g., planes 145, 445) of one or more tiles (e.g., tiles 140) of one or more die (e.g., 135), of one or more APM devices (e.g., APM device 125). In some embodiments, the inferences may be determined by circuitry (e.g., logic circuits) included in the APM devices. In these embodiments, the inferences may be provided as an output to an APM controller (e.g., APM controller 120) and/or a memory (e.g., memory 130). The APM controller may provide the inferences as an output to a host device (e.g., host device 105). In some embodiments, the results may be provided as an output to the APM controller and/or memory and the APM controller may make the inferences from the results. The APM controller may provide the inferences as an output to the host device 105. In some embodiments, the results may be provided as an output from the APM controller to the host device. The host device may then make the inferences based on the received results (e.g., using one or more processors or other computing devices).

The inferences may be made based on the result responsive to control signals (e.g., signals provided from the host to the APM controller) and/or responsive to the execution of computer-executable instructions (e.g., code of a software program) that implement the inferences. In some examples, the computer-executable instructions are stored in a non-transitory computer readable medium, such as memory 130 and/or a memory located on the host device. In some examples, the inferences may be made based on the result using hardware instead of or in addition to computer-executable instructions. For example, at least some of the operations for the inferences may be hardwired as one or more logic circuits included in an APM system and/or a host device.

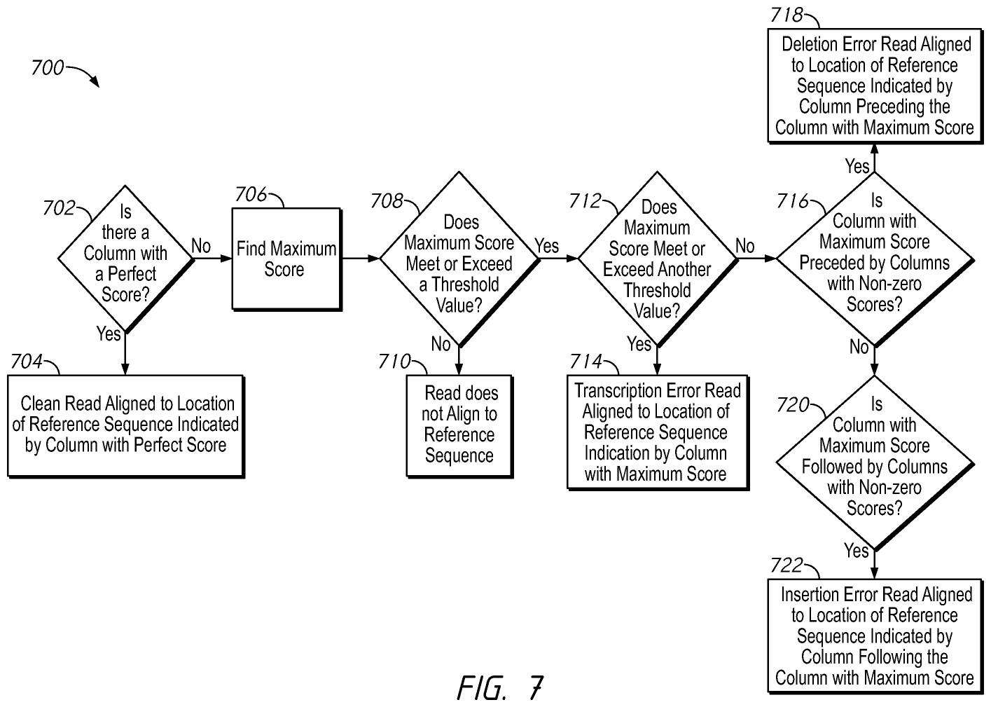

FIG. 7 is a flowchart illustrating inferences based on results for a read provided by an associated processing memory device in accordance with examples disclosed herein. For example, the inferences may be based on a comparison of a read to a reference sequence and its permutations stored in an APM device as shown in FIGS. 4 and 5. The APM may be APM device 125 in some examples. The results may include results 305 and/or 505 in some examples. The inferences may be made by one or more components of an APM system (e.g., APM system 110) and/or a host device (e.g., host device 105) in some examples.

As indicated by block 702 of flowchart 700, it may be determined whether one of the values in the result indicates a column (e.g., columns 150, 450) has a perfect score. That is, all of the nucleotides of a read match all of the nucleotides of the reference sequence (or its permutation) in the column. If yes, as shown by block 704, it may be inferred that the read is a clean read that is aligned to a location of the reference sequence indicated by the column associated with the perfect score.

If no, the maximum score (e.g., maximum value of the values included in the result) may be determined as indicated by block 706. Alternatively, in some examples, the maximum score may be determined and then analyzed to determine whether the maximum score is equal to the perfect score.

In some examples, such as the one shown in FIG. 7, the maximum score may be compared to a threshold value, as indicated by block 708. Based on the comparison, it may be determined whether the read aligns to the reference sequence. In the example shown in FIG. 7 and indicated by block 710, when the maximum score does not meet or exceed a threshold value, it may be determined that the read does not align to the reference sequence. This may be due to a variety of factors, including but not limited to, the sample from which the read was acquired is a different organism than that of the reference sequence, the sample from which the read was acquired has more mutations than tolerated, the read has more errors than tolerated, or a combination thereof.

If maximum score meets or exceeds the threshold value, it may be compared to another threshold value as indicated by block 712. However, in some examples, block 708 may be omitted and block 712 may be performed after the maximum score is determined in block 706. Based on the comparison to the other threshold value, it may be determined whether the read has a transcription error. In the example shown in FIG. 7, if the maximum score meets or exceeds the other threshold value, it may be determined the read is a transcription error read aligned to a location of the reference sequence indicated by the column with the maximum score as shown in block 714.

If the maximum score does not meet or exceed the other threshold value, the values of columns nearby (e.g., adjacent to) the column having the maximum score may be analyzed. As indicated by block 716, the values of the nearby columns may be analyzed to determine whether the column with the maximum score is preceded by columns with non-zero scores (e.g., at least one nucleotide matches between the read and the reference sequence). If yes, the read is determined to be a deletion error read aligned to a location of the reference sequence indicated by the column preceding the column with the maximum score as indicated by block 718.

If no, as indicated by block 720, it the values of the nearby columns may be analyzed to determine whether the column with the maximum score is followed by columns with non-zero scores. If yes, the read is determined to be an insertion error read aligned to a location of the reference sequence indicated by the column following the column with the maximum score as indicated by block 722. While block 716 is shown as preceding block 720, in some examples, the determination of block 720 may be performed prior to block 716. In some examples, the determinations of blocks 716 and 720 may be performed concurrently, at least in part.

In some examples, after making the inferences as illustrated in FIG. 7, the component making the inferences may provide an output that includes the alignment location and/or the type of error of the read (or that the read is clean if no error inferred).

While the examples of inferences are based on values representing a number of matching nucleotides, similar inferences may be based on values representing Hamming distances. For example, differences in Hamming distances between columns may be used to make similar inferences to differences in the number of matching nucleotides. In some examples, the considerations of the inferences may be inverse as Hamming distances increase as similarity decreases (whereas the number of matches decreases as similarity decreases). For example, rather than having a minimum value for a threshold value, a maximum Hamming distance may be provided (e.g., columns having a Hamming distance equal to or greater than a threshold value may be deemed not a match).

Although the examples herein describe storing one or more reference sequences in APM device(s) and providing reads to the APM device(s) for comparison, reads may also be stored in the APM device(s). For example, one or more reads may be stored in plane 445, and one or more reference sequences (or portions thereof) may be provided for comparison to the reads in plane 445. In some examples, various permutations of the reference sequence (or portions thereof) may be provided for comparison (e.g., shifted versions). Thus, instead of a read being compared to multiple permutations of a reference sequence, multiple reference sequences, and/or a combination thereof in parallel, a reference sequence may be compared to multiple reads in parallel. Further, instead of different reads being provided in series, different reference sequences, permutations of the reference sequence, and/or portions thereof may be provided in series.

Additionally, while the examples describe shifting the reference sequence by one nucleotide for each permutation, other permutations may be used for the reference sequence. For example, the reference sequence may be shifted by two nucleotides for each permutation. Similar inferences may be made, but with an appropriate adjustment in the threshold values and locations of the column distributions. Finally, while the examples describe DNA sequences, it is understood that the principles may be used for other sequences such as RNA sequences.

While the examples herein refer to determining “correct” locations of reads and/or alignment locations of reads for a reference sequence based on perfect scores and/or inferences, the locations within the reference sequence determined from the results may be candidate locations (may also be referred to as estimated or potential locations) locations for the reads. Genomic sequences may include regions where patterns of nucleotides are repeated. Thus, there may be several perfect and/or close matches for locations in the reference sequence where a read may be aligned. The chance of multiple candidate locations increases as the length of the read decreases and/or the length of the reference sequence increases.

After inferences have been made, as described herein, the APM device, APM controller, other component of the APM system, and/or host device may perform additional processing to “narrow down” the potential alignment locations of reads provided by the inferences when there are multiple potential alignment locations. In some applications, this may be based on one or more probabilistic methods known in the art of genetic sequencing. However, by using parallel processing capabilities of APM devices and/or systems as well as making the inferences based on the results provided by the APM devices and/or systems as disclosed herein, the overall computing time for aligning reads to reference sequences may be reduced.

Certain details set forth herein provide a sufficient understanding of examples of the disclosure. However, it will be clear to one having skill in the art that examples of the disclosure may be practiced without these particular details. Moreover, the particular examples of the present disclosure described herein should not be construed to limit the scope of the disclosure to these particular examples. In other instances, well-known circuits, control signals, timing protocols, and software operations have not been shown in detail in order to avoid unnecessarily obscuring the disclosure. Additionally, terms such as “couples” and “coupled” mean that two components may be directly or indirectly electrically coupled. Indirectly coupled may imply that two components are coupled through one or more intermediate components.

From the foregoing it will be appreciated that, although specific embodiments of the disclosure have been described herein for purposes of illustration, various modifications may be made without deviating from the spirit and scope of the disclosure. Accordingly, the scope disclosure should not be limited any of the specific embodiments described herein.

What is claimed is:

1. A method comprising:
storing, in a first column of a plane of an associative processing memory device (APM), a first genetic sequence, wherein individual rows of a plurality of rows in the first column store data corresponding to nucleotides of the first genetic sequence;
storing, in a subsequent plurality of columns of the plane of the plane, a corresponding plurality of permutations of the first genetic sequence;
comparing, in parallel, a second genetic sequence to the first genetic sequence and the plurality of permutations of the first genetic sequence; and
providing a result for the plane based, at least in part, on the comparing.

2. The method of claim 1, further comprising generating the plurality of permutations by shifting the first genetic sequence by one nucleotide per column for each one of the subsequent plurality of columns.

3. The method of claim 2, further comprising padding at least one row of the plurality of rows of the subsequent plurality of columns with data corresponding to “don't cares.”

4. The method of claim 3, wherein a number of the rows of the plurality of rows padded with “don't cares” corresponds to a number of nucleotides by which the first genetic sequence is shifted.

5. The method of claim 1, further comprising:
comparing, in parallel, a third genetic sequence to the first genetic sequence and the plurality of permutations of the first genetic sequence; and
providing a second result for the plane based, at least in part, on the comparing.

6. The method of claim 1, wherein providing the result comprises providing a plurality of values, wherein a number of plurality of values is equal to a number of the first column and the subsequent plurality of columns.

7. The method of claim 1, further comprising:
storing, in a second column of a second plane, a third genetic sequence, wherein individual rows of a second plurality of rows in the second column store data corresponding to nucleotides of the third genetic sequence; and
storing, in a second subsequent plurality of columns of the second plane of the second plane, a corresponding plurality of permutations of the third genetic sequence.

8. The method of claim 7, wherein the first genetic sequence and the third genetic sequence are corresponding portions of a plurality of portions of a reference sequence.

9. The method of claim 8, wherein the first genetic sequence and the third genetic sequence overlap by at least one nucleotide.

10. The method of claim 7, wherein the plane and the second plane are located on different tiles of the APM device, the method further comprising:
comparing, in parallel, the second genetic sequence to the first genetic sequence, the plurality of permutations of the first genetic sequence, the third genetic sequence, and the plurality of permutations of the third genetic sequence; and
providing a second result for the second plane based, at least in part, on the comparing.

11. A method comprising:
comparing, with an associative processing memory (APM) device, a first genetic sequence to a second genetic sequence and a plurality of permutations of the second sequence stored in the APM device; and
determining an alignment location of the first genetic sequence in the second genetic sequence based on the comparing.

12. The method of claim 11, wherein the second genetic sequence and the plurality of permutations of the second genetic sequence are stored in separate ones of a plurality of columns of the APM device, wherein the APM device provides a result based on the comparing, the result comprising a plurality of values, individual ones of the plurality of values corresponding to the plurality of columns, wherein each of the plurality of values indicates a number of matching nucleotides between the first genetic sequence and the second genetic sequence.

13. The method of claim 12, wherein determining the alignment location comprises:
determining a maximum value of the plurality of values; and
comparing the maximum value to a perfect score, wherein when the maximum value is equal to the perfect score, the alignment location is determined to be a location of the second sequence indicated by a column associated with the maximum value.

14. The method of claim 12, wherein determining the alignment location comprises:
determining a maximum value of the plurality of values;
comparing the maximum value to a perfect score; and
comparing the maximum value to a threshold value when the maximum value is less than the perfect score,
wherein the alignment location is determined to be a location of the second sequence indicated by a column associated with the maximum value when the maximum value is equal to or greater than the threshold value.

15. The method of claim 14, further comprising determining an error in the first genetic sequence, wherein the error is determined to be a transcription error.

16. The method of claim 12, wherein determining the alignment location comprises:
determining a maximum value of the plurality of values;
comparing the maximum value to a perfect score; and
comparing the maximum value to a threshold value when the maximum value is less than the perfect score,
when the maximum value is less than or equal to the threshold value, determining whether one or more columns nearby a column associated with the maximum value are associated with non-zero values of the plurality of values.

17. The method of claim 16, wherein the alignment location is determined to be in a first column preceding the column associated with the maximum value, when the one or more columns nearby associated with the non-zero values precede the column associated with the maximum value.

18. The method of claim 17, further comprising determining an error in the first genetic sequence, wherein the error is determined to be a deletion error.

19. The method of claim 16, where the alignment location is determined to be in a first column following the column associated with the maximum value, when the one or more columns nearby associated with the non-zero values follow the column associated with the maximum value.

20. The method of claim 19, further comprising determining an error in the first genetic sequence, wherein the error is determined to be an insertion error.

21. The method of claim 12, determining the alignment location comprises:
determining a maximum value of the plurality of values;
comparing the maximum value to a threshold value; and
determining the first genetic sequence does not align to the second genetic sequence when the maximum value is less than or equal to the threshold value.
